(19) United States
(12) Patent Application Publication
(10) Pub. No.: US 2004/0209320 A1
(43) Pub. Date: Oct. 21, 2004

(54) Humate production

(76) Inventors: Jeremiah L. Newcomb, San Diego, CA (US); Ronald A. Newcomb, San Diego, CA (US)

Correspondence Address: FISH & RICHARDSON, PC, 12390 EL CAMINO REAL, SAN DIEGO, CA, 92130-2081, US

(21) Appl. No.: 10/414,870

(22) Filed: Apr. 15, 2003

(51) Int. Cl. C12P 1/00

(52) U.S. Cl. 435/41

(57) ABSTRACT

A method of producing humate compositions involves environmentally friendly and cost-effective methods of soaking the starting materials in a large quantity of water in order to leech out the humates. The starting materials are lignocellulose based, and the reaction is driven by the addition of fungi, manganese and air. The resulting compositions have a wide variety of applications in the industrial, environmental, agricultural and health fields.

### TECHNICAL FIELD

This invention relates to improved processes for producing humates, and more particularly to specific processes resulting in faster production of concentrated humic substances.

### BACKGROUND

Humates are ubiquitous in the environment, however their exact structural configuration remains a mystery. Humic acid, for example, is not a simple chemical species, but a designation for a group of very large molecules. Humic substances are known to interact with a wide variety of elements including nutrients, toxic metals, radionuclides and halogens. Humic substances can complex metals, sequester anthropogenic organic compounds, oxidize and reduce elements to and from toxic forms, photosensitize chemical reactions and enhance or retard the uptake of toxic compounds or micronutrients to plant and microbial organisms. As more is learned about humates, new uses are likely to be developed in industrial, environmental, agricultural and health fields, among others.

Traditional humate production involves its extraction from non-renewable substances such as coal or shale or soil based substances. These processes often involve use of highly acidic or highly alkaline reagents. Recently, there has been increased focus on alternative starting materials and processes of producing humate substances. These newer processes often employ as starting materials lignocellulose-based waste products, such as paper pulp. Production involves environmentally friendly and cost-effective methods of soaking the starting materials in a large quantity of water in order to leech out the humates from the lignins.

### SUMMARY

Improved processes for the production of liquids containing a high gravimetric value of lignocellulase-based humates have now been developed. These environmentally friendly processes result in faster extraction of the humic substances and higher concentrations of humates recovered per starting material.

The details of one or more embodiments of the invention are set forth in the description below. Other features, objects, and advantages of the invention will be apparent from the description and from the claims.

### DETAILED DESCRIPTION

Lignins are complex natural phenyl polymers with many various but deliberate couplings. The specific, highly variable chemical structures are determined within various plant types. Lignins are formed by dehydration of plant sugars produced by the process of photosynthesis. Depending upon the type of plant sugar that has been formed by the specific plant, the dehydration process produces a variety of lignin monomers, which in turn form diverse lignin polymers. The polymers are branched and cross-linked to form three-dimensional structures. During the recovery process, the phenyl groups break into non-specific chains with attached organic acid groups. These complex molecules having variable structures are generally grouped together under terms such as “humates,” “humic acid,” and “humic substances,” for example.

Humate substances include, but are not limited to acids and bases of humin, humic acid, ulmic acid, hymetomelanic acid, fulvic acid, tannins, phenolic compounds, polyphenols, phenolic aralkyl compounds, depsides, associated benzenoid compounds, for example, and metal analogues, chelates solids, precipitates and solutions of these compounds, all of which may be treated to produce various analogs and isolates of the individual substances.

Any lignin containing substance may be used as a starting material in the process. Fibrous plant materials are preferred. Waste materials are especially attractive as being an environmentally friendly choice. Examples of lignin containing starting materials include, but are not limited to pulps, pulp washes, recycled paper, waste paper, straw, brans, grains, vegetable fibers, vegetable stalks, kelp, wood fibers, wood chips, sawdust, wood flakes, landscaping waste, such as twigs and leaves, animal manures, and combinations thereof. Other suitable starting materials are readily apparent to any person having skill within this art.

The lignin containing material(s) may be used directly as obtained, or pre-processed to provide maximum benefits for reaction speed, efficiency and recovery. For example, the lignin containing materials may treated to increase surface area, such as mechanically, for example, by shredding, chopping, grating or grinding. Other processes that aid in the break down of lignins also may be useful, such as application of ultraviolet and/or microwave energy and/or subjecting the materials to chemical processes selected for their ability to break down the lignins. Such processes may influence the final composition by resulting in humates of smaller sizes. It should be appreciated that the higher the content of lignin in the starting material, the smaller amount of material is needed in order to produce a complete reaction, i.e., wherein the resulting liquid is saturated with humates.

The raw or pre-processed lignin containing materials are added to a reaction vessel. The vessel should allow entry of air or oxygen to the reaction. This may be achieved, for example, by selecting a vessel that is open at least at one portion, or a closed vessel adapted to accept at least one inlet of air or oxygen. Optionally, one or more outlets may be present for emission of carbon dioxide produced during the reaction.

Water is added to the reaction vessel. While the amount of water is not critical, it should be appreciated that a preferred amount of water covers the starting materials and is present in a volume large enough to accept the humates without premature saturation. Generally, the ratio of water to starting materials is at least about 2:1 by volume. The water may be from any fresh source, such as tap water, well water, water from lakes, ponds, streams and rivers, and purified and deionized water.

Fungi should be present in the mixture. Fungi may naturally be present in the starting materials. Alternatively, one or more types of fungi may be added in amount of about one teaspoon of dried, active fungi powder per 100 gallons of reactants. Useful fungi are those with the ability to digest sugars. Ascomycetes (or Ascomycota), and Basidiomycetes (or Basidiomycota) phyla are most preferred for their ability to digest saccharides, lignin, lignose, lignone, or lignireose and/or several complex phenyl compounds containing lignin fractions such as Ligno Sulfinate or Lignosol (an alcohol form). Most useful fungi would be considered yeasts. The fungi produce large and varied groups of enzymes useful to the production of humates, such as peroxidases and monooxygenases. The presence of a wide range of enzymes allows for the presence of other free radicals that can aid in driving the reaction. Greater numbers and varieties of enzymes available to react with any given lignin analogue will result in production of greater numbers and varieties of humate analogues.

Manganese also should be present in the mixture in an amount sufficient to drive the reaction. Manganese is available as salt powders. Manganese powder(s) may be added in an amount of about one half teaspoon per 100 gallons of reactants. One or more ingredients optionally may be used in addition to the manganese. Such ingredients include but are not limited to calcium, iodine, iron, potassium, sodium and nitrogen. These ingredients may be made more soluble, for example, by using salt forms. These ingredients are useful even in trace amounts as the fungi use them for growth, thus resulting in faster reactions. Because humates are known to aid plant uptake of manganese as well as the optional ingredients, any of those ingredients remaining in the liquid after completion of the reaction would be beneficial if the humate containing liquid were used for plant products and processes.

Because the process is aerobic, air and/or oxygen should be introduced to the reaction. This may be accomplished by simply performing the reaction in an open vessel. Alternatively, air and/or oxygen may be introduced to the reaction via periodic or constant agitation, such as stirring. Most preferred is air and/or oxygen introduction via one or more inlets that allows the gas(es) to bubble through the reaction mixture. Multiple aeration methods may be employed. The gas(es) may be introduced throughout the entire reaction or at intervals. The gas(es) may be introduced after an initial period of reaction without their addition, for example after about one to about three days. Optimal timing and amount of the addition of gas(es) will depend upon the nature and amount of the starting materials, the type(s) of fungi present, the size of the reaction vessel, and the volume of liquid, and can be determined through routine experimentation. Since the reaction produces acids, which slow oxidation, adding air or oxygen increases pH by the reduction of carbon dioxide.

The reaction may be performed at room temperature. Optionally, slight heating or cooling may be performed, especially for the purposes of slowing or speeding the reaction. Determination of optimum temperatures to be used for desired results can be easily assessed by anyone having ordinary skill within the art.

The initial pH of the reactant mixture is generally in the range from about 7.5 to about 9.5, typically in the range from about 8.0 to about 9.0. As the reaction takes place, the pH will drop significantly. By the end of the reaction, the pH is generally in the range from about 5.2 to about 7.2, typically in the range from about 5.7 to about 6.7. Depending upon the nature of the starting materials and the type(s) of fungi present, preferred pH ranges may be determined by routine experimentation and may be maintained though the use of common reagents.

The reaction is completed when the liquid is saturated with humates or when the increase in concentration of humates in the liquid does not significantly increase over time. Typically, the reaction takes about six to about seven days for 100 gallons of mixture reacting at room temperature. The resulting liquid should have the appearance of black tea or coffee.

The liquid may be separated from the solids by conventional methods, such as draining, extraction, pressing, filtering, and/or sieving, either with or without the aid of pressure. If the reaction resulted in a saturated liquid, the solid materials may be recovered and used as starting materials for additional reactions until they become depleted of the ability to produce humic substances.

The resulting liquid optionally may be concentrated, pasteurized, frozen, freeze-dried and/or fortified with ingredients beneficial to the overall usefulness of the liquid. The choice of additional beneficial ingredients depends upon the desired end use. For example, where the liquid is prepared for use as a plant fertilizer, optional ingredients such as nitrogen, phosphorous, potassium, iron, calcium, silver, zinc, vanadium, selenium, rubidium, manganese, or chromium, may be added to either the initial starting materials or to the resulting liquid. The liquid may also be useful for nutrition, wherein additional ingredients such as vitamins, trace elements, herb derivatives and tannins may provide additional beneficial effects. Such optional ingredients are appreciated and known by people having ordinary skill within the pertinent art.

The concentrated humate liquid has applications in the agricultural, horticultural and hydroponic fields, especially as an algaecide and crop stimulant. Additionally, it has applications in sealife, animal and human nutrition, especially for nutrient assimilation. Humates also are useful for the removal of organic contaminants, heavy metals, and polycyclic aromatic hydrocarbons, especially in water, where the humates can serve as flocculants. Humates are used in many additional applications, including the production of certain polymers. These and other applications for humic substances are known in the art. Further uses for humates are currently being developed, and others await discovery. The liquid may be post-processed in order to produce the form that is most suitable for the desired use, e.g., liquid or powder, and then used in all applications that involve the use of humates.

A number of embodiments of the invention have been described. Nevertheless, it will be understood that various modifications may be made without departing from the spirit and scope of the invention. Accordingly, other embodiments are within the scope of the following claims.

What is claimed is:

1. A process for the production of a composition comprising humates, the process comprising the steps of:
a) combining at least one lignin containing substance, water, fungi and manganese to form a reaction mixture;
b) aerating the reaction mixture; and
c) separating the resulting liquid from the solids.

2. The process according to claim 1, further comprising the step of pre-processing the at least one lignin containing substance.

3. The process according to claim 2, wherein the pre-processing step is selected from the group consisting of size reduction, application of ultraviolet energy, application of microwave energy, chemical treatment, mechanical processing, and combinations thereof.

4. The process according to claim 1, wherein the manganese is in salt form.

5. The process according to claim 4, wherein the manganese salt is present in an amount of about one half teaspoon per 100 gallons of reaction mixture.

6. The process according to claim 1, further comprising the step of adding heat to the reaction mixture.

7. The process according to claim 1, further comprising the step of removing heat from the reaction mixture.

8. The process according to claim 1, further comprising the step of adjusting the pH of the reaction mixture in a range from about 5.2 to about 9.0.

9. The process according to claim 1, further comprising the step of adding at least one ingredient selected from the group consisting of calcium, iodine, iron, potassium, sodium, nitrogen and salts and combinations thereof.

10. The process according to claim 1, further comprising the step of concentrating the resulting liquid.

11. The process according to claim 1, further comprising the step of pasteurizing the resulting liquid.

12. The process according to claim 1, wherein the solids are used for more than one reaction process.

13. The process according to claim 1, wherein the process is carried out in a reaction vessel having at least one open portion.

14. The process according to claim 1, wherein the process is carried out in a closed reaction vessel having at least one inlet source of an oxygen containing gas.

15. The process according to claim 14, wherein the closed reaction vessel comprises at least one gas outlet.

16. The process according to claim 1, wherein aeration is performed during the entire process.

17. The process according to claim 1, wherein aeration is performed intermittently.

18. The process according to claim 1, wherein aeration is delayed until after the reaction has been partially completed.

19. The process according to claim 1, wherein the ratio of starting material to water is at least about 1:2 by volume.

20. The process according to claim 1, wherein the ratio of water to starting material is at least about 2:1 by volume.

21. The process according to claim 1, wherein the at least one lignin containing substance is selected from the group consisting of pulps, pulp washes, recycled paper, waste paper, straw, brans, grains, vegetable fibers, vegetable stalks, kelp, wood fibers, wood chips, sawdust, wood flakes, twigs, leaves, animal manures, and combinations thereof.

22. The process according to claim 1, wherein the water is obtained from a source selected from the group consisting of tap, well, lake, pond, stream, river, purified, deionized and combinations thereof.

23. The process according to claim 1, wherein the fungi is selected from the group consisting of Ascomycetes, Basidiomycetes and combinations thereof.

24. The process according to claim 1, wherein the fungi is selected for its ability to digest lignin compounds selected from the group consisting of lignin, lignose, lignone, lignireose, lignin containing phenyl compounds, saccharides and combinations thereof.

25. The process according to claim 1, wherein the fungi is one or more yeasts.

26. The process according to claim 1, wherein the amount of fungi is about one teaspoon of fungi powder per 100 gallons of reaction mixture.

27. In a process for producing humates, the improvement being the combination of the addition of manganese and aeration to the reaction.

28. A composition made by the process according to claim 1.
